(19) United States
(12) Patent Application Publication
(10) Pub. No.: US 2007/0297617 A1
(43) Pub. Date: Dec. 27, 2007

(54) Neighbor friendly headset: featuring technology to reduce sound produced by people speaking in their phones

(76) Inventor: Thomas G. Cehelnik, Tucson, AZ (US)

Correspondence Address: Thomas G. Cehelnik, 8300 E. Ocotillo Dr., Tucson, AZ, 85750, US

(21) Appl. No.: 11/473,426

(22) Filed: Jun. 23, 2006

(51) Int. Cl. A61F 11/06; H04R 1/32

(52) U.S. Cl. 381/71.6

(57) ABSTRACT

A method and apparatus is disclosed for reducing the annoying sound produced by people speaking into the phone while in public. The technology uses the headset microphone to sample the sound at the location of the headset and uses a loudspeaker on the headset to cancel the sound produced by the talker at that point and beyond in the surrounding environment. A novelty of the invention is the loudspeaker feedback to the headset microphone produced by the speaker is necessarily cancelled from the microphone channel.

### CROSS REFERENCE TO RELATED APPLICATIONS

A Mobile Phone Extension patent applications by the author provide background for establishing the connection between microphone and loudspeaker of a headset.

### STATEMENT OF FEDERALLY SPONSORED RESEARCH OR DEVELOPMENT

Not applicable

### REFERENCE SEQUENCE LISTING OR COMPUTER PROGRAM

Sequence listing is not applicable

### FIELD OF THE INVENTION

This invention relates to audio apparatus and audio communication technology such as used in phones handset and headset and audio recorders and audio players such as the popular IPOD of Apple Computer.

### BACKGROUND OF THE INVENTION

The use of wireless communication audio devices, particularly the use of cell phone wireless headsets such as the popular BlueTooth headset technology, has results in many people speaking into their microphone while in public. The user's speech makes for annoying background noise. To help with this noise pollution problem, the technology presented here reduces the sound level coming from the phone user.

### APPROACH

The use of a headset is ideal for using sound cancellation theory to reduce noise of people talking. This is because the headset contains a microphone to sample the sound pressure. What is needed is a loudspeaker to play an equal but opposite sound pressure away from the headset. However, to do this requires that the loudspeaker signal is cancelled from the microphone signal before it is sent to the cell phone. A method is disclosed for taking this approach.

### KEY FEATURES OF INVENTION

A key capability of the invention is to reduce sound level heard by neighboring people when someone is speaking on their cell phone.

Also it is possible to mix in outside sound to play in the ear so when people are not on the phone, they will be able to still hear what is going on around them.

### BRIEF DESCRIPTION OF DRAWINGS

The invention will be better understood upon reading the following Detailed Description while referencing the provided drawings.

FIG. 1 shows a person with a wireless Bluetooth headset equipped with a speaker that sends out sound of opposite phase to that received by the microphone.

FIG. 2 shows the wireless headset having a microphone [6] and loudspeaker [8] used for sound cancellation. View a) shows sideview, and view b) showed front end view.

FIG. 3 shows the signal paths to allow for the processing.

### DETAILED DESCRIPTION OF THE INVENTION

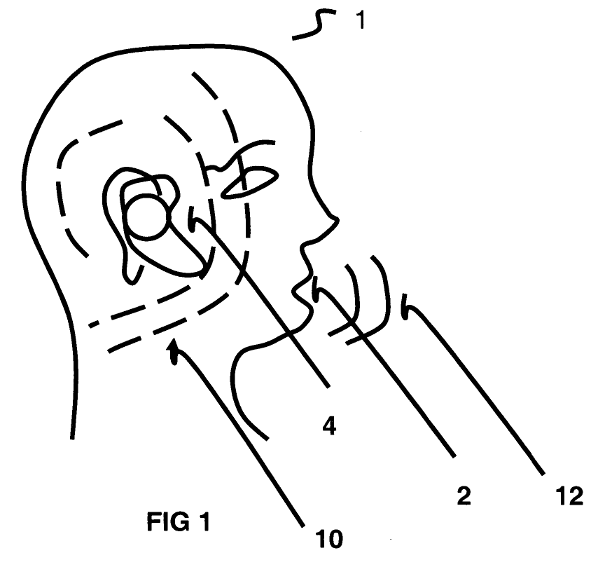

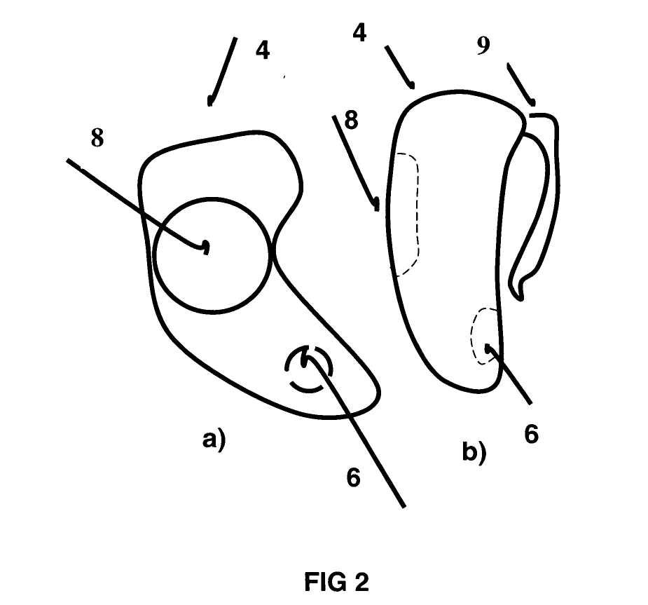

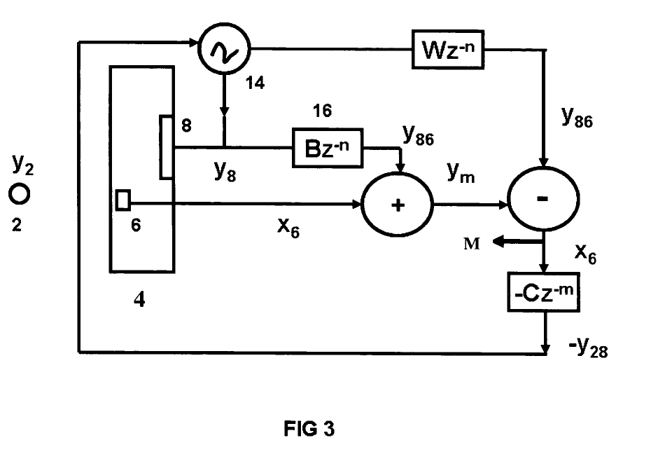

FIG. 1, FIG. 2 are presented to show the physical application and hardware. FIG. 3 is used to show the signal processing to result in the sound cancellation. All figures are needed as the discuss that follows uses the labels of the items in those figures.

The physical idea is the sound [12] is in the form of a signal y₂ produced by the mouth [2]. When this sound propagates to the location of the loudspeaker [8] it has been delayed in time and modified by diffraction, to yield a signal y₈. The sound radiating past the ear is reduced by playing the loudspeaker [8] so it generates and acoustical signal of opposite phase and equal amplitude to y₈.

The law of superposition applies to sound waves, so the net signal is reduced at the location [8]. The signal is also reduced at farther away points because the sound y₈ continues outward away from the user but so does the signal −y₈ from the loudspeaker. This is to say the sound y₈ is effectively generated by a point source. The loudspeaker [8] then collocates a point source of equal and opposite amplitude.

In detail the microphone and loudspeaker are not exactly located at the same location, thus there is a time delay, and possibly some amplitude changes that occur as the sound travels between the microphone location and the loudspeaker location. The time delay of n-time samples is represented in FIG. 3, by the z⁻ⁿ, and the amplitude by the transfer function B.

This transfer function B and time delay n is measured in our method by sending white noise through the loudspeaker [8] and capturing signals in the microphone when there is no speech [12]. This is a novelty, along with the fact that reciprocity demands that the same transfer function applies to sound passing from the microphone location to the loudspeaker location. Hence, we know how the sound [12] is modified as it passes the microphone to the speaker, and we know how the sound is modified after it is generated by the loudspeaker [8] and sent to the microphone [6].

It is noted that those with familiarity in signal processing will note that z is the Z-transform representation, and B is really a filter transfer function. In the time domain the output of a filter is the convolution with the filter function. Same nomenclature applies for other filters W, and C in the diagram.

The transfer function B and its delay is computed as the autocorrelation of the received microphone signal divided by the autocorrelation of the transmitted signal. The transmitted signal should be white noise over the audio bandwidth of the speech, that is roughly up to 100-3500 Hz. Reducing the bandwidth will still result is some noise reduction, as it is conceivable that in some cases just certain frequencies may be desirable to cancel due to hardware and cost limitations and body diffraction effects.

FIG. 3 shows the signal coming from the microphone [6] is mixed with the electrical signal from the sound of the loudspeaker [8]. Another novelty, is this distortion is removed by subtracting off the feedback y₈₆ to give a clean microphone signal x₆ to be sent out to the cell phone from point M. Ideally, we see that the transfer function W would be equal to B. Also the delays would be identical, as shown in the FIG. 3 with z⁻ⁿ for each path.

Variations in electrical paths and hardware and additive noise can cause differences. The cancellation can be done either by analog circuitry or by digital processing. Cehelnik, has presented a well performing analog circuit in his Cell Phone Extention patent application.

To do the processing digitally, noise cancellation techniques are used. The optimum filter W is found by adjusting its taps to minimize the expectation of the output power in the signal x₆. The adjustment can be nonadaptive such as by using a Weiner filter, or adaptive algorithms such as the Least Mean Squares algorithm, LMS, are possible.

There are various ways to break up the problem as a signal filter, or multiple filters using the measured filter B and its corresponding delay. Since we measure this response, as a option for the user of the device, a good estimate of B and its delay are found. This makes the filter W have to work less hard, and it is conceivable that it may only have to adjust amplitude and delay slightly. Thus the processing is minimal by measuring the transfer function B and its corresponding delay, since W will be close to same value as B. W may have to introduce a slight delay, but most of it is already captured in the y₈₆ leg by the z⁻ⁿ factor.

The next significant feature is the generation of the antiphase signal −y₂₈. This is done by again recognizing that the transfer function is C, is ideally what we already have measured, and this is the default value. This can be tweaked for optimum cancellation by setting in the filter in the factory. The optimum filter is determined by measuring the radiated sound level produced by a model head speaking into the headset. The minus sign is just a negative multiply.

The capture of the signals to allow signal processing are done in the headset hardware where an analog to digital converter is used to digitize the microphone audio as normal. The mixed in signals are generated digitally to make the subtraction through digital signal processing in a computer or digital signal processing chip within the heads set. Other means also exist for the processing such as through a wireless network when speed allows. Once a digital representation of the signal −y₂₈ is formed it is played into the loudspeaker with a digital to analog converter DAC. The amplification and phase is adjusted accordingly so the desired amplitude and phase of the output audio signal is that of −y₂₈, the opposite of the audio signal [12] arriving at the loudspeaker location [8] from the mouth of the talker [2].

It is also noted as useful, that by switching the role of the speaker and the microphone as described, we can listen to the environment and then play the sound back to users ear. This way, there is not loss in hearing of the surrounding sounds when the user wears the headset. The sound cancellation to eliminate feedback can also be used. This feature is useful and not address by current products on the market.

1. A method of making an audio handset or headset that reduces the user's sound level in the surroundings by having:
a. Speaker to generate antiphase audio sent away from the body;
b. A measured transfer function between the said loudspeaker and the microphone;

2. A method of including a microphone to receive surrounding sounds on a headset for use with cell phone and audio headsets to help make it safer for people to hear when they are not using the phone or their audio device but are wearing the headset;
